(19) United States
(12) Patent Application Publication
(10) Pub. No.: US 2005/0144707 A1
(43) Pub. Date: Jul. 7, 2005

(54) Goggles for water-related activities

(76) Inventor: Chih-Ming Chen, Tainan City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/750,952

(22) Filed: Jan. 5, 2004

(51) Int. Cl. A61F 9/02

(52) U.S. Cl. 2/426

(57) ABSTRACT

A pair of goggles includes a frame and a wearing portion. The frame includes at least one loop portion for receiving a lens unit. The lens unit has an isolated empty chamber for providing the pair of goggles with buoyancy and thus enabling the pair of goggles to float on water. In an embodiment, the lens unit includes two lenses and an airtight spacing loop securely sandwiched between the lenses, thereby defining the isolated empty chamber. In another embodiment, the frame has only one loop portion, and the lens unit includes two lenses of one-piece type and an airtight spacing loop securely sandwiched between the lenses, thereby defining the empty chamber.

### BACKGROUND OF THE INVENTION

1. Field of the Invention

The present invention relates to a pair of goggles for water-related activities. In particular, the present invention relates to a pair of goggles that is buoyant.

2. Description of the Related Art

Water-related activities includes aquatic activities and activities proceeded ashore, such as fishing, swimming, diving, water volleyball, water ballet, surfing, water motorcycling, etc. Most people would wear a pair of goggles with normal lenses or correcting lenses (in a case that the wearer suffers from myopia, hyperopia, and/or astigmatism) while proceeding with water-related activities to shield the eyes from the sunlight and/or to prevent water from entering the eyes. It is not uncommon that the pair of goggles falls and thus sinks into the water when proceeding with a vigorous activity.

FIG. 1 of the drawings illustrates a pair of conventional goggles 1 including a frame 1 and two temples 12 that can be replaced by a head strap when appropriate. The frame 1 includes two ring portions 111 for respectively receiving two solid lenses 112. The densities of the frame 1 and lenses 112 are much greater than that of water. Thus, it is difficult to find the pair of goggles 1 if it sinks in deep water. One would have to either ask for help for searching the pair of goggles or stop the activity. Otherwise, the activity has to be continued in an inconvenient or even risking way, as injury may be caused.

### SUMMARY OF THE INVENTION

In accordance with an aspect of the present invention, a pair of goggles includes a frame and a wearing portion. The frame includes at least one loop portion for receiving a lens unit. The lens unit has an isolated empty chamber for providing the pair of goggles with buoyancy and thus enabling the pair of goggles to float on water.

In an embodiment of the invention, the lens unit includes two lenses and an airtight spacing loop securely sandwiched between the lenses, thereby defining the isolated empty chamber.

In another embodiment of the invention, the frame has only one loop portion, and the lens unit includes two lenses of one-piece type and an airtight spacing loop securely sandwiched between the lenses, thereby defining the empty chamber.

Other objects, advantages, and novel features of the invention will become more apparent from the following detailed description when taken in conjunction with the accompanying drawings.

### BRIEF DESCRIPTION OF THE DRAWINGS

FIG. 1 is a perspective view, partly exploded, of a pair of conventional goggles.

FIG. 2 is a perspective view, partly exploded, of a pair of goggles in accordance with the present invention.

FIG. 3 is a perspective view of the pair of goggles in accordance with the present invention.

FIG. 4 is a sectional view taken along plane 4-4 in FIG. 3.

FIG. 5 is a perspective view, partly exploded, of a modified embodiment of the pair of goggles in accordance with the present invention.

FIG. 6 is a schematic view illustrating buoyancy of the pair of goggles in accordance with the present invention.

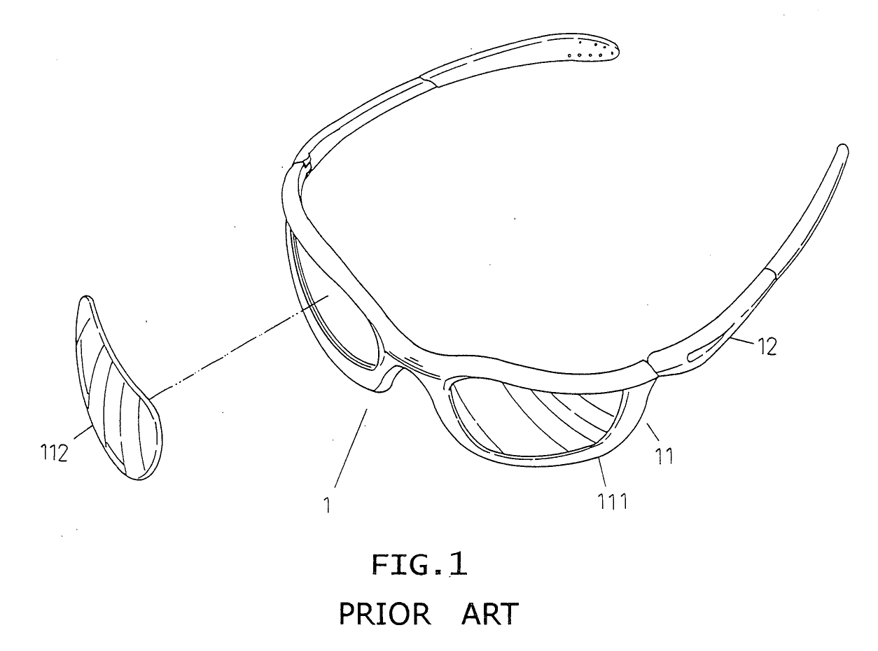

### DETAILED DESCRIPTION OF THE PREFERRED EMBODIMENTS

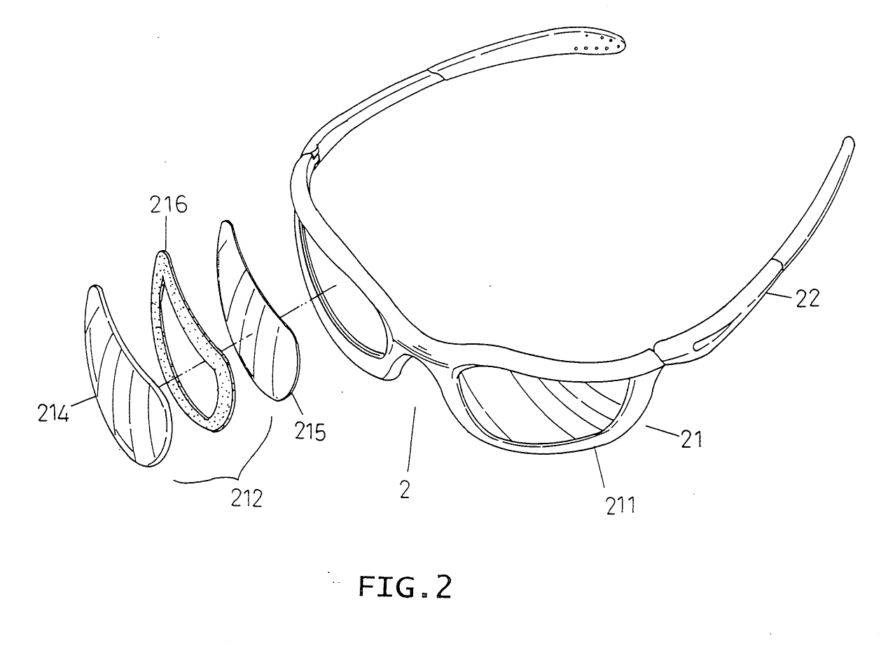

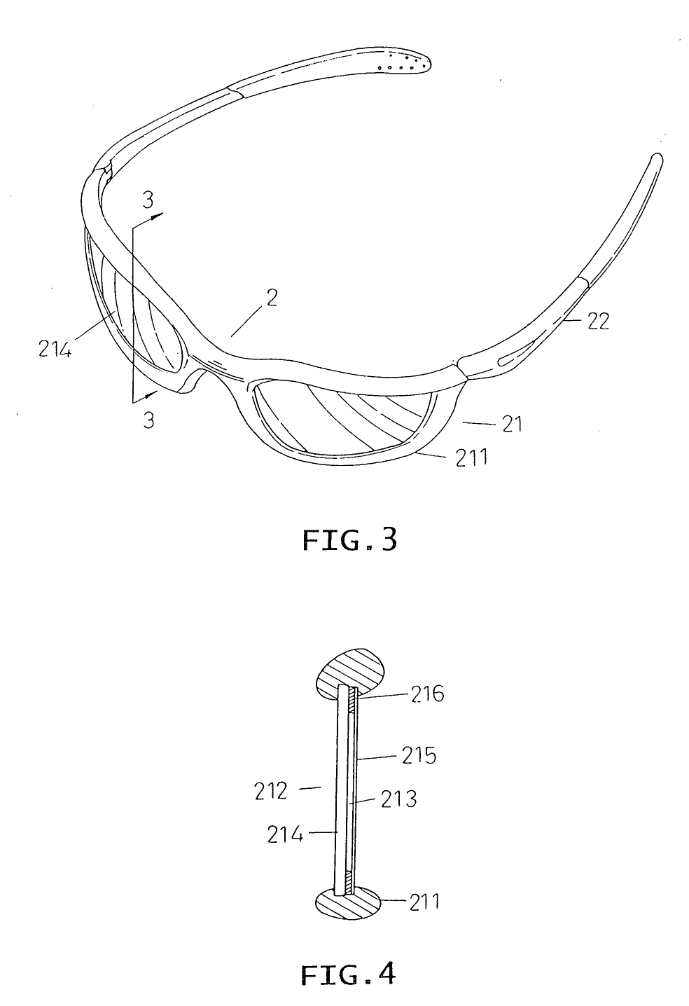

Referring to FIGS. 2 through 4, a pair of goggles in accordance with the present invention is designated by 2 and generally comprises a frame 21 and a wearing portion 22. The wearing portion 22 includes a pair of temples or a head strap that allows a user to put the pair of goggles on. The pair of goggles in a broad meaning includes a pair of glasses, a pair of sunglasses, etc.

The frame 21 includes at least one ring portion or loop portion for receiving at least one lens unit 212. In this embodiment, the frame 21 includes two loop portions 211 for respectively receiving two lens units 212. Each lens unit 212 includes an isolated empty chamber 213 defined therein. In this embodiment, each lens unit 212 includes two lenses 214 and 215 and an airtight spacing loop 216 securely sandwiched between the lenses 212, thereby defining the empty chamber 213. Two sides of the spacing loop 216 are respectively bonded to the lenses 214 and 215 without the risk of permeation of water. The pair of goggles is buoyant due to provision of the empty chamber 213 that is isolated from the outside. In an alternative embodiment, the lens unit 212 includes an integral lens with an empty chamber 213 formed by means of blowing molding.

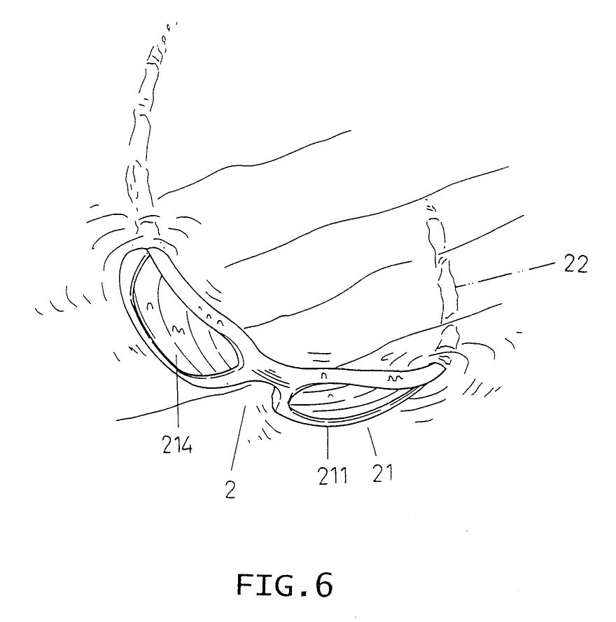

Referring to FIG. 6, when proceeding with a water-related activity, the pair of goggles in accordance with the present invention floats on the water if the pair of goggles falls into the water. Thus, the wearer can find it easily.

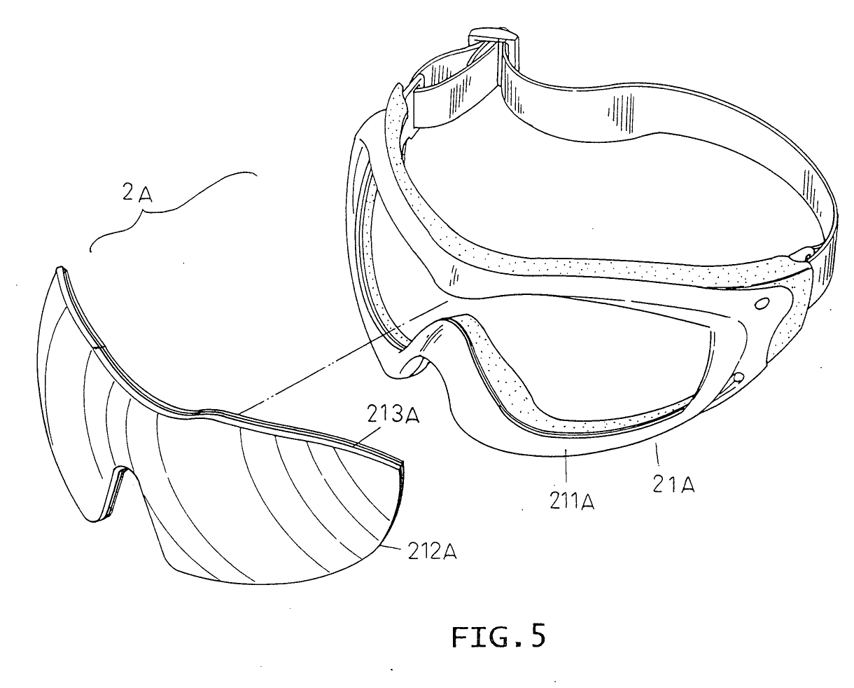

FIG. 5 illustrates a modified embodiment of the present invention. In this embodiment, the pair of goggles (now designated by 2A) includes a frame 21A having a loop portion 211A in which a lens unit 212A is received. The lens unit 212A includes an isolated empty chamber 213A. Similar to the above embodiment, the lens unit 212A includes two lenses of one-piece type and an airtight spacing loop 216 securely sandwiched between the lenses without the risk of permeation of water.

Although the invention has been explained in relation to its preferred embodiment, it is to be understood that many other possible modifications and variations can be made without departing from the scope of the invention as hereinafter claimed.

1. A pair of goggles comprising a frame and a wearing portion, the frame including at least one loop portion, at least one lens unit being received in said at least one loop portion, said at least one lens unit including an isolated empty chamber for providing the pair of goggles with buoyancy and thus enabling the pair of goggles to float on water.

2. The pair of goggles as claimed in claim 1, wherein said at least one lens unit includes two lenses and an airtight spacing loop securely sandwiched between the lenses, thereby defining the isolated empty chamber.

3. A pair of goggles comprising a frame and a wearing portion, the frame including a loop portion, a lens unit being received in the loop portion, the lens unit including an isolated empty chamber for providing the pair of goggles with buoyancy and thus enabling the pair of goggles to float on water.

4. The pair of goggles as claimed in claim 3, wherein the lens unit includes two lenses of one-piece type and an airtight spacing loop securely sandwiched between the lenses, thereby defining the empty chamber.
